(19) United States
(12) Patent Application Publication
(10) Pub. No.: US 2024/0033472 A1
(43) Pub. Date: Feb. 1, 2024

(54) IN-SITU FENESTRATION DEVICES WITH ARTICULATING ELEMENTS

(71) Applicant: Medtronic Vascular, Inc., Santa Rosa, CA (US)

(72) Inventors: Mark L. STIGER, Santa Rosa, CA (US); Keith D. PERKINS, Santa Rosa, CA (US); Steven Michael CLAESSENS, Santa Rosa, CA (US)

(21) Appl. No.: 18/227,376

(22) Filed: Jul. 28, 2023

Related U.S. Application Data
(60) Provisional application No. 63/393,054, filed on Jul. 28, 2022.

(51) Int. Cl. A61M 25/00; A61F 2/95

(52) U.S. Cl. CPC A61M 25/0043; A61F 2/95; A61F 2002/9534

(57) ABSTRACT

An in-situ fenestration device including a fenestration catheter extending along a longitudinal axis and a proximal tip extending from the fenestration catheter. The fenestration catheter includes a first articulating element and a second articulating element. The first articulating element is articulable about the longitudinal axis from a delivery state to a deployment state. The second articulating element articulable about the longitudinal axis from the delivery state to the deployment state. The first articulating element and the second articulating element in the deployment state are configured to form a fenestration in a graft material at a fenestration site of a stent graft.

### CROSS-REFERENCE TO RELATED APPLICATION

This application claims the benefit of U.S. provisional application Ser. No. 63/393,054 filed Jul. 28, 2022, the disclosure of which is hereby incorporated in its entirety by reference.

### TECHNICAL FIELD

The present disclosure relates to in-situ fenestration devices with articulating elements (e.g., elliptical articulating elements).

### BACKGROUND

In-situ fenestration (ISF) has seen limited applicability to aortic stent grafts for endovascular aneurysm repair (EVAR) and thoracic endovascular aneurysm repair (TEVAR). In-situ fenestration of aortic stent grafts can be used to maintain perfusion to blood vessels (e.g., aortic side branch arteries or peripheral arteries) located in an area excluded by a stent graft. In-situ fenestration may be used to fenestrate (e.g., create a new opening or hole) in a stent graft in-situ (e.g., in the place of the stent graft) following deployment of the stent graft within a vascular system. Application of ISF has been typically limited to removing unintentional coverage of blood vessels (e.g., arteries) upon deployment of a stent graft, but has rarely been used in elective scenarios.

### SUMMARY

In one embodiment, an in-situ fenestration device including a fenestration catheter extending along a longitudinal axis and a proximal tip extending from the fenestration catheter is disclosed. The fenestration catheter includes a first articulating element and a second articulating element. The first articulating element is articulable about the longitudinal axis from a delivery state to a deployment state. The second articulating element articulable about the longitudinal axis from the delivery state to the deployment state. The first articulating element and the second articulating element in the deployment state are configured to form a fenestration in a graft material at a fenestration site of a stent graft.

In another embodiment, an in-situ fenestration device is disclosed. The in-situ fenestration device includes a fenestration catheter extending along a longitudinal axis and a proximal tip extending from the fenestration catheter. The fenestration catheter includes a distal element articulable the longitudinal axis from a delivery state to a deployment state. The fenestration catheter includes a proximal element articulable the longitudinal axis from the delivery state to the deployment state. The distal element has a distal element delivery cross sectional profile in the delivery state relative the longitudinal axis and a distal element deployment cross sectional profile in the deployment state relative the longitudinal axis. The distal element deployment cross sectional profile is larger than the distal element delivery cross sectional profile. The proximal element has a proximal element delivery cross sectional profile in the delivery state relative the longitudinal axis and a proximal element deployment cross sectional profile in the deployment state relative the longitudinal axis. The proximal element deployment cross sectional profile is larger than the proximal element delivery cross sectional profile. The distal element and the proximal element in the deployment state are configured to form a fenestration in a graft material at a fenestration site of a stent graft.

In yet another embodiment, a method of forming a fenestration in a graft material at a fenestration site of a stent graft is disclosed. The method includes delivering a fenestration device to the fenestration site. The fenestration device includes a fenestration catheter extending along a longitudinal axis and a proximal tip extending from the fenestration catheter. The fenestration catheter including first and second articulating elements. The method further includes deploying the fenestration device at the fenestration site by positioning the first and second articulating element on first and second sides of the graft material and articulating the first and second articulating elements to form spaced apart first and second articulated elements. The method further includes contacting the spaced apart first and second articulated elements with the graft material to form contacting first and second articulated elements. The method also includes applying heat to at least one of the contacting first and second articulated elements to disassociate the graft material from the stent graft at the fenestration site to form the fenestration.

### BRIEF DESCRIPTION OF THE DRAWINGS

FIG. 1A depicts a partial cut away, schematic, side view of an abdominal aorta and right and left renal arteries extending therefrom where a stent graft excludes the right and left renal arteries from blood perfusion.

FIG. 1B depicts a partial cut away, schematic, side view of an aortic arch branching into a brachiocephalic artery, a left common carotid artery, and a left subclavian artery where a stent graft excludes the left subclavian artery from blood perfusion.

FIG. 2 depicts a schematic view of an in-situ fenestration system according to one embodiment.

FIG. 3A depicts a perspective, cross section view of an in-situ fenestration system according to one embodiment.

FIG. 3B depicts a side, cross section view of the in-situ fenestration system shown in FIG. 3A.

FIG. 3C depicts a perspective view of the in-situ fenestration system of FIG. 3A during a cutting operation.

FIGS. 4A through 4H depict side, cross section views of the in-situ fenestration system of FIG. 3A in different stages of an in-situ fenestration cutting operation according to one embodiment.

FIGS. 5A through 5D depict an alternative graft material gripping mechanism according to one or more embodiments for gripping graft material.

FIGS. 6A through 6E depict an embodiment with an articulating laser catheter used to perform a cutting operation of an in-situ fenestration process.

FIGS. 7A and 7B depict a distal portion of a laser catheter having first and second rings of discrete ports for laser light at a distal end thereof.

FIGS. 7C through 7F depict an alternative embodiment where a steerable catheter is configured to impart a vacuum to grip graft material aligned with the ostium of a branch vessel.

FIG. 8 depicts a schematic, cross section view of an aortic arch with a branch vessel branching therefrom where an in-situ fenestration device is deployed to form a fenestration in the graft material of a stent graft.

FIGS. 9A through 9D depict schematic, side views of an in-situ fenestration device performing a cutting operation on the graft material of a stent graft.

FIGS. 9E and 9F depict schematic, side views of in-situ fenestration device and deployment of embolic protection device therefrom.

FIGS. 10A through 10G depict schematic, side views of an in-situ fenestration device and graft material in relation to procedural steps for implementing the in-situ fenestration device.

FIGS. 11A, 11B, and 11C are schematic, top views of a cutter assembly including distal cutters and proximal cutters in a delivery configuration, a first deployment configuration, and a second deployment configuration, respectively.

FIG. 12 depicts a schematic, side view of a stent graft deployed in an aortic arch to bridge an aneurysm, and a branch stent graft disposed in the brachiocephalic artery branching from the aortic arch.

FIG. 13 depicts a schematic, side view of a deflectable catheter accessing a stent graft through the left subclavian artery.

FIG. 14 depicts a schematic side view of a fenestration catheter advanced through the deflectable catheter.

FIG. 15 depicts a schematic side view of a fenestration catheter where a proximal portion and a distal portion are in a closed configuration, thereby trapping graft material therebetween.

FIG. 16 depicts a schematic side view of a branch stent graft secured to a fenestration formed by a fenestration catheter.

FIGS. 17A through 17N depict schematic side views of a fenestration catheter having first and second elliptical articulating elements connected to the fenestration catheter in various stages of procedural deployment within a branch vessel branching from a main vessel having a deployed stent graft therein.

FIGS. 18A and 18B depict plan views of a fenestration formed by the fenestration catheter.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system the terms “distal” and “proximal” are used in the following description with respect to a position or direction relative to a treating clinician. “Distal” and “distally” are positions distant from or in a direction away from the clinician, and “proximal” and “proximally” are positions near or in a direction toward the clinician. For the stent-graft prosthesis, “proximal” is the portion nearer the heart by way of blood flow path while “distal” is the portion of the stent-graft further from the heart by way of blood flow path.

The following detailed description is merely exemplary in nature and is not intended to limit the invention or the application and uses of the invention. Although the description is in the context of treatment of blood vessels such as the aorta, coronary, carotid, and renal arteries, the invention may also be used in any other body passageways (e.g., aortic valves, heart ventricles, and heart walls) where it is deemed useful.

In-situ fenestration (ISF) has seen limited applicability to aortic stent grafts for endovascular aneurysm repair (EVAR) and thoracic endovascular aneurysm repair (TEVAR). In-situ fenestration of aortic stent grafts can be used to maintain perfusion to blood vessels (e.g., aortic side branch arteries or peripheral arteries) located in an area excluded by a stent graft. In-situ fenestration may be used to fenestrate (e.g., create a new opening or hole) in a stent graft in-situ (e.g., in the place of the stent graft) following deployment of the stent graft within a vascular system. Application of ISF has been typically limited to removing unintentional coverage of blood vessels (e.g., arteries) upon deployment of a stent graft, but has rarely been used in elective scenarios.

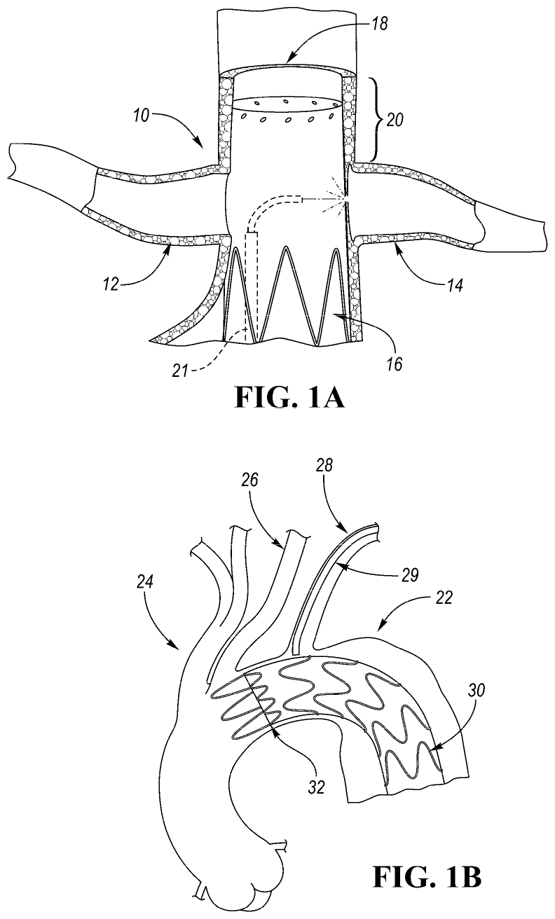

FIG. 1A depicts a partially cut away, schematic, side view of abdominal aorta 10 and right renal artery 12 and left renal artery 14 extending from abdominal aorta 10. Right and left renal arteries 12 and 14 may be referred to generally as the renal arteries. Stent graft 16 includes proximal end 18 and a distal end (not shown). Proximal end 18 of stent graft 16 lands in landing zone 20 of abdominal aorta 10. Stent graft 16 extends from landing zone 20 to exclude perfusion to right renal artery 12 and left renal artery 14. An in-situ fenestration at the exclusion areas (e.g., using laser fenestration device 21) can be used to perfuse right renal artery 12 and left renal artery 14. Perfusion may result from blood flow through the fenestration alone or through a branch stent graft inserted into the fenestration after it is created and extending into the branch artery.

FIG. 1B depicts a partial cut away, schematic, side view of aortic arch 22 branching into brachiocephalic artery 24, left common carotid artery 26, and left subclavian artery 28. Brachiocephalic artery 24, left common carotid artery 26, and left subclavian artery 28 may be referred to generally as side branch arteries. Stent graft 30 includes proximal end 32 and a distal end (not shown). Stent graft 30 extends to exclude perfusion to left subclavian artery 28. An in-situ fenestration (e.g., using laser fenestration device 29) at the exclusion area created at left subclavian artery 28 can be used to perfuse left subclavian artery 28 (e.g., via the fenestration or a later-deployed branch stent graft).

In-situ fenestration may provide a solution for implementing stent grafts with patients having hostile neck anatomy within their abdominal aorta. Current stent graft seal technology is unsuitable for many aortic anatomies. Many aortic abdominal and thoracic aortic aneurysms present either a relatively short seal zone (e.g., 0 to 10 millimeters) and/or a high degree of landing zone angulation. Examples of such anatomies include a short neck aneurysm, no neck thoraco-abdominal aneurysm, reverse conical neck, and highly angled aneurysm neck with a short landing zone inner curve. Under these circumstances, an alternative landing zone may be used that excludes perfusion to peripheral arteries (e.g., the renal arteries). In-situ fenestration may be used to open these excluded areas to permit blood perfusion. However, adequate in-situ fenestration processes and related devices/systems have not been proposed to realize the potential of in-situ fenestration in this regard.

Accordingly, clinicians (e.g., doctors or physicians) have investigated other techniques for modifying stent grafts for EVAR and TEVAR patients. The existing techniques (e.g., dedicated off-the-shelf multibranch devices, custom-made multibranch devices, clinician modified devices, and peripheral techniques) do not adequately modify stents grafts to completely address blood perfusion.

For instance, dedicated off-the-shelf multibranch devices may have low patient applicability due to variability in the anatomy of patients. The geometry to accommodate multiple branches on a dedicated branch device can be complicated to determine. Procedures to deploy these devices are complex. Branching canulation and/or stenting can be complicated because the devices are susceptible to rotational or axial misalignment.

An alternative technology is a custom-made multibranch device. However, these devices require a significant lead time (e.g., 6 to 8 weeks) and are not available for emergent cases. Moreover, custom ordered devices may still be susceptible to axial and rotational misalignment.

Clinicians have modified stent grafts themselves before deploying the stent graft in the vascular system of the patient. Physicians can partially deploy an off-the-shelf stent graft on a sterile field and make fenestrations based on patient specific anatomy. This type of “back table” modification of an off-the-shelf stent graft may have one or more benefits. Radio frequency (RF) or thermal energy (e.g., eye cautery) may be used to clean and/or seal any frayed and/or cut fiber ends at the fenestration boundary. The size of the fenestration is customizable without post dilation, which may cause material damage. The fenestrations can be made using three-dimensional (3D) reconstructions from patient specific computed tomography (CT) scans. The fenestrations can be reinforced with sutures and/or guidewires to make a durable interface between the main stent graft and the branch stent graft. However, these procedures include unloading of the stent graft so that it can be modified with a fenestration. Reloading the stent graft is a challenge due to the low profile and high packing density of the stent graft in the radially compressed, delivery state. These modifications are typically labor and time intensive.

Techniques for providing blood flow to peripheral blood vessels used in connection with off-the-shelf stent grafts have also been proposed. Clinicians can deploy off-the shelf stent grafts in parallel with these techniques to permit blood perfusion to peripheral arteries and respective organs. Examples of these types of technologies chimneys, snorkels, and sandwich techniques. A chimney structure may be applied in the abdominal aorta and may include a renal chimney and a seal zone distal to a lower chimney. A different structure may be applied in the aortic arch where blood flows into a chimney from the aortic arch and blood flows out of the chimney into the left common carotid artery, and blood flows into a periscope from the aortic arch and blood flows out of the periscope into the left subclavian artery. Another technique is referred to as a sandwich. Blood flows into the celiac artery and superior mesenteric artery (SMA) from sandwich parallel chimneys. These techniques may have one or more of the following benefits: (1) available for emergent cases; (2) configurations can be adapted for patient-specific anatomies (e.g., ballerina techniques); and/or (3) planning using 3D reconstructions from patient specific CT scans. However, these techniques have durability concerns and potential mid or long-term occlusion risks relating to challenging hemodynamics.

Due to one or more drawbacks of the existing technologies identified above, there has been interest in developing in-situ fenestration technology that addresses one or more of the drawbacks identified above. In-situ fenestration encompasses processes in which apertures are made in a fully or partially deployed stent graft inside of a patient. Under limited circumstances, in-situ fenestration has been employed to provide perfusion in the aortic arch, the visceral segment, and the iliac arteries. In the aortic arch, in-situ fenestration can be made in a retrograde direction (e.g., outside of the stent graft) using supra-aortic access. Other anatomies may use in situ fenestration using an antegrade technique (e.g., inside the stent graft). In-situ fenestration may have one or more of the following benefits: (1) provides a multibranch solution independent of patient anatomical constraints thus providing for a larger applicability; (2) can be performed using off-the-shelf stent grafts; and/or (3) may avoid time-consuming “back-table” modification and technically challenging reloading into delivery systems.

However, current in-situ fenestration techniques suffer from one or more drawbacks. Current in-situ fenestration methods result in relatively small size apertures where aggressive post-dilation is used to accommodate a branch stent graft. Needle in-situ fenestration uses a needle to create an initial fenestration. Laser fenestration uses a laser ablation catheter having a diameter of 2.0 to 2.5 millimeters. Radio frequency (RF) ablation may also be used. One example of an RF ablation method uses a 0.035 inch powered wire. As a drawback, damage to the graft material during fenestration expansion adds to procedural variability and makes durability testing difficult. Additionally, lack of standardized protocols results in lack of consistency in fenestrations, thereby inhibiting consistent anticipation of intermediate and long-term durability.

In one or more embodiments, in-situ fenestration process and/or related devices are disclosed that at least partially addresses one or more of the following drawbacks and/or at least partially provides one or more of the following benefits. A potential drawback of existing technology is anatomical variability limiting patient applicability of dedicated off-the shelf branch devices. A potential benefit of in-situ fenestration is customization of off the shelf stent grafts that is independent of anatomical constraints. Custom devices have been proposed but take a relatively long time (e.g., 6-8 weeks) for manufacture and deliver, and may not be available for emergent cases. A potential benefit of in-situ fenestration is application to off-the-shelf devices with no manufacturing or shipping delays.

Another potential drawback relates to “back table” modification of off-the-shelf devices by clinicians. These modified devices are difficult to reload, limiting adoption of this method. In-situ modification of a stent graft occurs in-situ, and thereby eliminating the step of reloading the device into a delivery system. Custom and “back table” modified devices are susceptible to axial or rotational misalignment which can impact vessel canulation. Fenestrations created in-situ after the deployment of a stent graft are independent of the position of the main graft.

Current in-situ fenestration procedure lack standardization in terms of initial fenestration source and post dilation procedures. A potential benefit of standardization would be the reduction or elimination of severe post dilation steps that can cause unpredictable damage to a graft material.

Current in-situ fenestration procedures may result in cut fibers and/or ripped material. These drawbacks may represent a source of procedural variability and may limit the long-term durability and seal of the fenestration and branch stent graft interface. One or more embodiments disclose a method for sealing cut fibers that help prevent continued breakdown of the fenestration and branch stent graft interface.

Current fenestration techniques start with a small initial fenestration that is aggressively post dilated to accommodate a branch graft which can result in the tearing of the graft material. Some graft materials use cutting balloons for post dilation, which may cause additional cut fibers and material damage. One or more embodiments disclose a method and/or device for forming a fenestration in-situ of a size and shape that involves little or no post dilation and/or cutting balloons.

Power sources (e.g., laser and RF ablation) for current in-situ fenestrations may create steam bubbles and generate char particles that can pose embolic risk. One or more embodiments disclose a method and/or device to allow in-situ fenestration creation while minimizing steam bubbles and char formation.

In one embodiment, an in-situ fenestration system is disclosed. The in-situ fenestration system includes a gripping device having a central channel and a fenestration device configured to advance along and through the central channel of the gripping device. The gripping device may be a two-piece arrangement configured to grip graft material therebetween. The fenestration device may be a steerable catheter having a distal portion configured to grip the graft material and including a cutter configured to cut the graft material. The cutter may be a laser configured to emit laser pulses.

One or more embodiment discloses a combination of a steerable gripping catheter and a laser catheter configured to locate, access, and perform an in-situ fenestration using a laser energy source, a vacuum for aspiration, and mechanical retrieval of the excised graft material. One or more visualization methods may be used to access a branch blood vessel via inner graft access with a steerable gripping catheter. A graft gripping mechanism may be used to hold and position a laser cutting head in a desired location. Once the graft material is brought into contact with the steerable gripping catheter, a laser catheter may be energized and guided through the graft wall. The size of the in-situ fenestration may be altered by altering a laser's cut radius via a deflectable portion of the laser catheter. The laser catheter may be configured to simultaneously create a fenestration in the implant material while cauterizing (e.g., melting or fusing) the edges of the fenestration. Vacuum aspiration may be applied during the laser cutting operation. The vacuum aspiration may sequester and remove heat and bubbles formed during the operation of creating the fenestration, or particulates and fibers. In an alternative embodiment, a deflectable portion of a laser catheter may determine a variation in the size of an intended fenestration, and a circular deflection of the laser catheter determines the area of the graft to be removed. Once the circular patch of the graft material has been detached, it may be extracted by a steerable gripping catheter, which initially pierced through the implant.

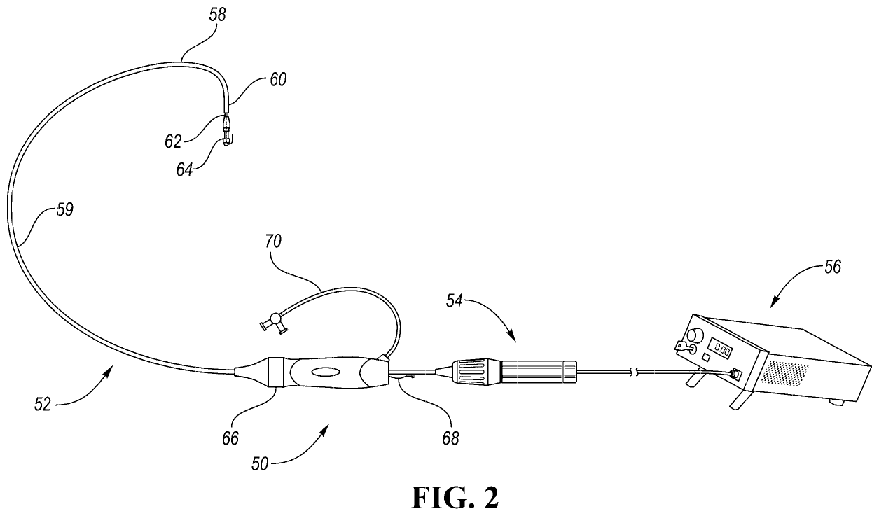

FIG. 2 depicts a schematic view of in-situ fenestration system 50 according to one embodiment. In-situ fenestration system 50 includes steerable gripper catheter 52, actuator handle 54, and laser power source 56. Steerable gripper catheter 52 includes flexible distal end region 58 configured to flex and bend a desired number of degrees (e.g., 90 degrees). Steerable gripper catheter 52 includes outer sheath 59. Radiopaque marker 60 may be included on flexible distal end region 58 and configured to provide visualization of the position of steerable gripper catheter 52 within a patient's vasculature. In-situ fenestration system 50 also includes inner catheter 62 including distal end 64 having features configured to grasp graft material and/or cut graft material with a laser (as further described below) and an actuator 54. Inner catheter 62 may be configured to be inserted into and through steerable gripper catheter 52, with distal end 64 extending out of the end of steerable gripper catheter 52 during a cutting operation. Steerable catheter flex actuator 66 is configured to flex flexible distal end region 58 from a straight, delivery position to an angled, deployment position. The degree of rotation of catheter flex actuator 66 may control the degree of curvature of flexible distal end region 58. In at least one embodiment, flexible distal end region 58 may bend at a 90 degree angle, or substantially thereabout, to engage the wall of the stent graft in a perpendicular configuration.

As shown in FIG. 2, in-situ fenestration system 50 also includes hemostasis valve 68 configured to track inner catheter 62, which may be configured to laser cut graft material at a fenestration site. Actuator handle 54 may be configured to be actuated such that distal end 64 grasps graft material. Actuator handle 54 may also be actuated to trigger cutting of graft material using a laser and deflecting the distal end 64. Laser power source 56 may be configured to supply laser power to distal end 64 of inner catheter 62 in connection with the cutting operation. As shown in FIG. 2, port 70 extends from steerable catheter flex actuator 66. Port 70 may be configured to perform flushing and/or aspirating operations in connection with one or more embodiments as disclosed herein.

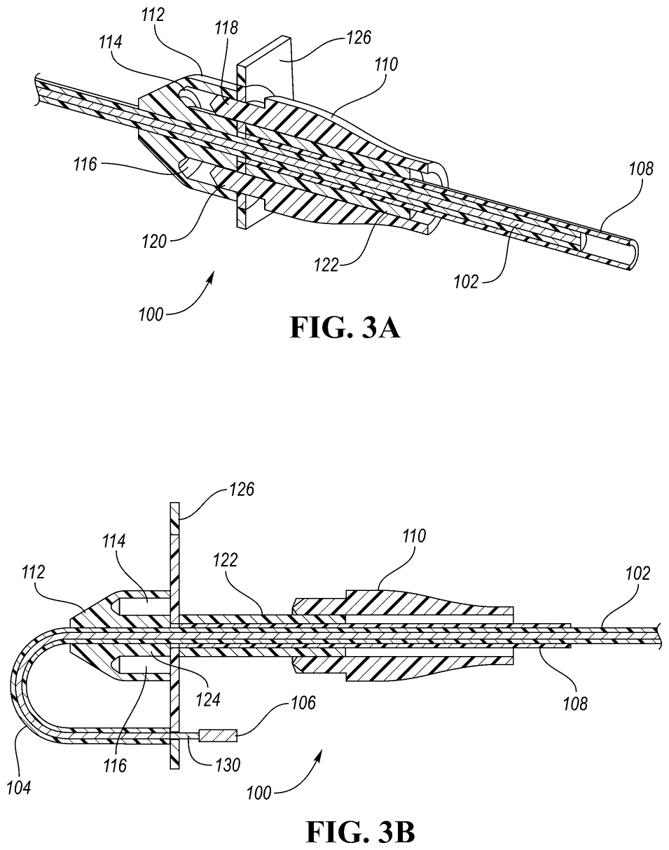

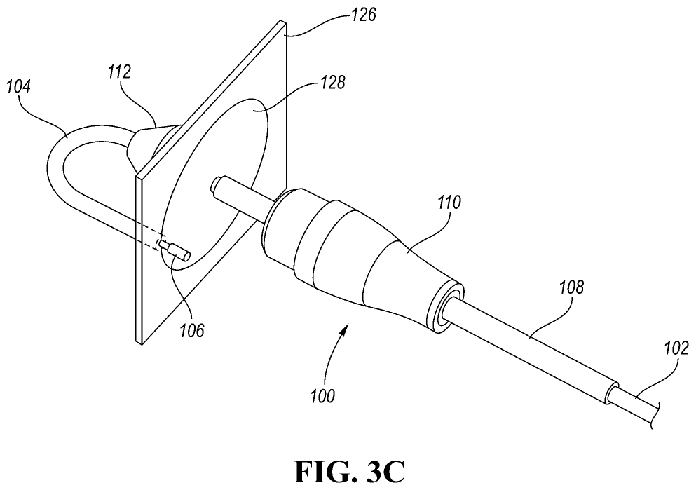

FIG. 3A depicts a perspective, cross section view of in-situ fenestration system 100 according to one embodiment. FIG. 3B depicts a side, cross section view of in-situ fenestration system 100. FIG. 3C depicts an in-situ fenestration system 100 during a cutting operation.

In-situ fenestration system 100 includes steerable catheter 102 having flexible distal portion 104 and distal portion 106 configured to enable emission of a laser pulse from a laser catheter. In another embodiment, distal portion 106 may be vibrated at a high frequency (e.g., ultrasonic) to help it pierce the graft material.

Steerable laser catheter 102 is configured to track within lumen 108 of a steerable gripper catheter. The laser may be included on the distal end of a laser catheter 102. Laser catheter may include a protector component (e.g., distal portion 106) configured to protect the laser tip. The protector component may be formed of platinum. The laser catheter may be formed of a polymeric material, such as a PEBAX polymeric material. The laser catheter system may also have a deflection actuator handle 54 configured to actuate the laser at the distal tip of the laser catheter. The actuator handle may be reusable.

In-situ fenestration system 100 also includes gripper capsule 110 and gripper head 112. Gripper capsule 110 is configured to translate (e.g., distal and proximal movement) relative to inner lumen 108 of the steerable gripper catheter. Gripper head 112 is also configured to translate (e.g., distal and proximal movement) relative to capsule 110 to grip the graft material prior to laser cutting and excising the graft material. Gripper capsule 110 and/or gripper head 112 may be formed of a plastic material. Gripper head 112, guide 122 and capsule 110 form the distal end of the steerable gripper catheter. An actuator mechanism 54 actuates flexing of distal portion 104 of the steerable laser catheter 102 and may be included in a handle. The handle may be formed of a plastic material. Gripper head 112 defines grooves 114 and 116 and gripper capsule 110 includes portions 118 and 120 configured to mate with groove 114 and 116.

In-situ fenestration system 100 also includes gripper backplate 122 situated between gripper capsule 110 and inner lumen 108. Gripper backplate 122 is configured to advance relative gripper capsule 110. Gripper head 112 includes stop 124 configured to prevent further advancement of gripper backplate 122. As shown in FIGS. 3A and 3B, gripper backplate 122 is in a gripping position to grip graft material 126 between gripper backplate 122 and gripper head 112. As shown in FIGS. 3B and 3C, flexible distal portion 104 of steerable laser catheter 102 is in a flexed position where distal portion 106 faces (e.g., is perpendicular to) graft material 126. Steerable laser catheter 102 is configured to rotate (e.g., 360 degrees) while the laser ray is being emitted from the laser catheter to cut circular-shaped fenestrated material 128 in graft material 126. The length of advancement of steerable catheter 102 relative to graft material 126 and/or the degree of flexing of distal portion 104 may be used to adjust the radius of the fenestration.

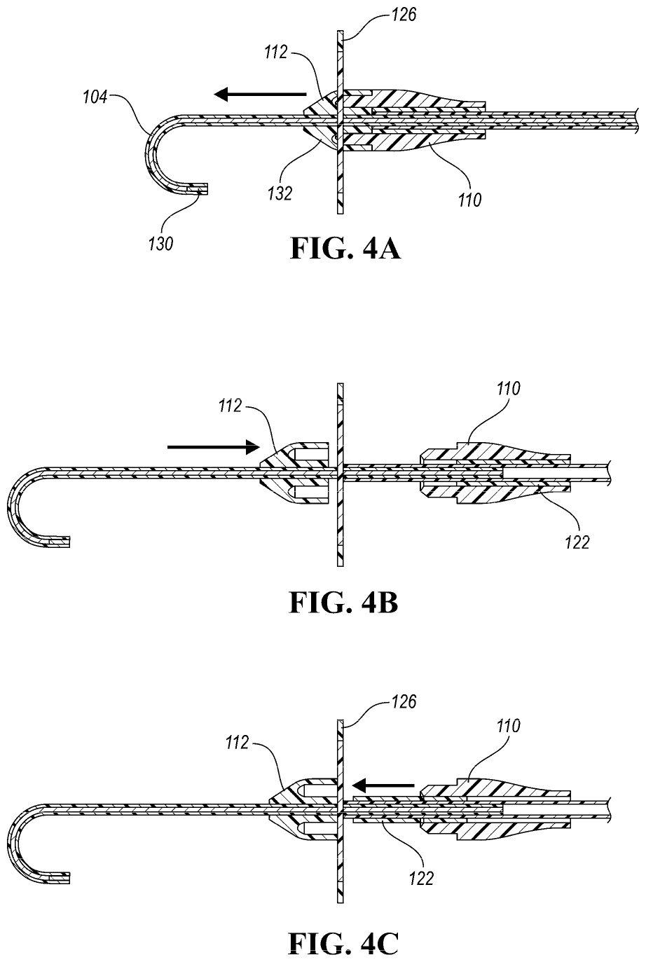

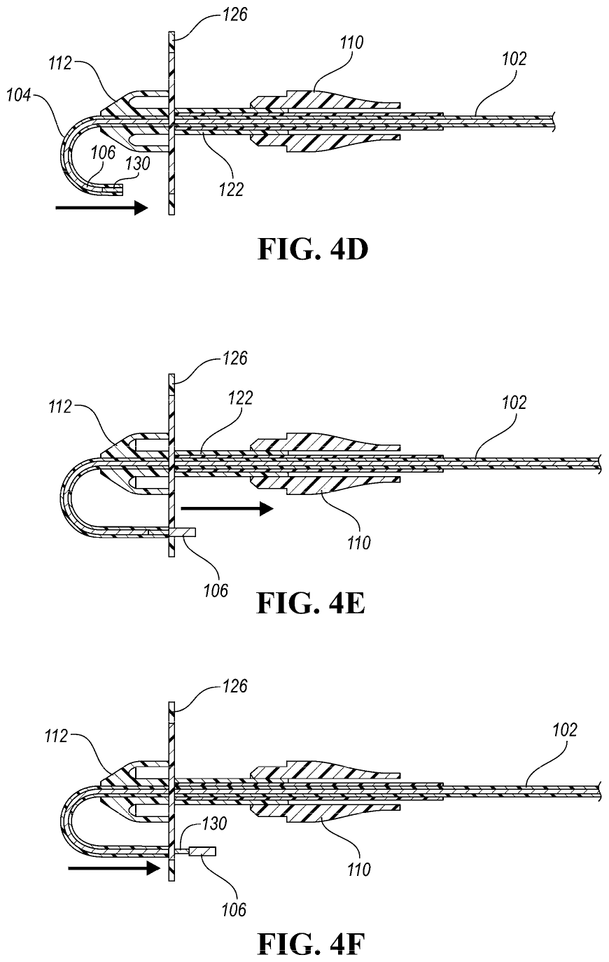

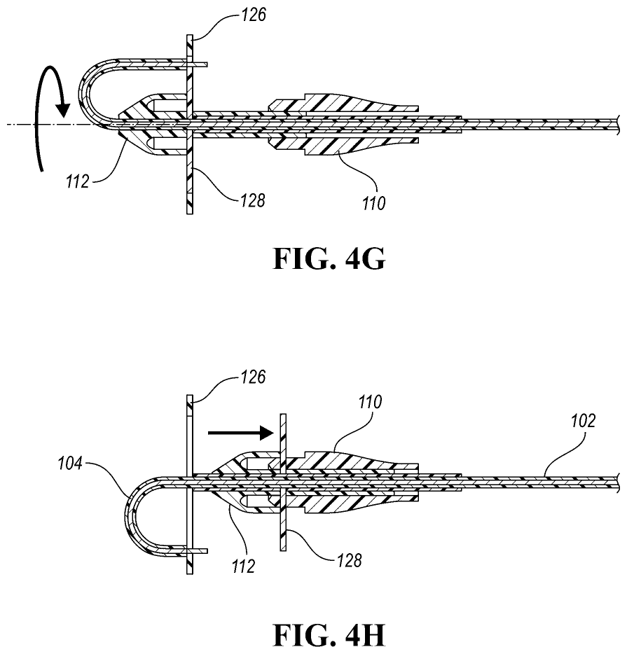

FIGS. 4A through 4H depict side, cross section views of in-situ fenestration system 100 in different stages of an in-situ fenestration cutting operation according to one embodiment. In one or more embodiments, the stages shown in FIG. 4A through 4H are sequential in timing during the cutting operation of in-situ fenestration system 100.

In the stage shown in FIG. 4A, gripper head 112 is advanced through graft material 126. Gripper head 112 may be advanced through a pilot aperture formed in graft material 126. The pilot aperture may be formed by laser tip 130 of a laser catheter. Alternatively, the pilot aperture may be formed by the distal end of distal portion 106 having a cutting feature (e.g., a sharpened or pointed tip that can cut through graft material). Gripper head 112 has a tapered distal end 132 configured to dilate the pilot aperture as gripper head 112 is advanced through graft material 126. As shown in FIG. 4A, grooves 114 and 116 of the gripper head 112 are engaged with portions 118 and 120 of the gripper capsule 110, as gripper head 112 is advanced through graft material 126. As gripper head 112 advances through graft material 126, flexible distal portion 104 of steerable laser catheter 102 is in an unflexed position aligned with the longitudinal axis of steerable catheter 102. Flexible distal portion 104 may have a portion extending beyond the distal end of gripper head 112 or may be retracted into the center channel of gripper head 112 during this operation. Once gripper head 112 advances through graft material 126 a desired distance, flexible distal portion 104 may be flexed into a flexed position as shown in FIG. 4A.

In the stage shown in FIG. 4B, gripper head 112 advances through graft material 126 to a position in which gripper head 112 is entirely on the other side of graft material 126. As gripper head 112 is clearing graft material 126, gripper capsule 110 and gripper backplate 122 may disengage from gripper head 112, by retracting gripper capsule 110 relative to gripper head 112, thereby forming a gap between the gripper capsule 110 and gripper head 112. At this stage, gripper backplate 122 is retracted within the channel of gripper capsule 110.

In the stage shown in FIG. 4C, gripper backplate 122 is advanced from within the channel of gripper capsule 110 into an advanced position where gripper backplate 122 closes the gap between the gripper capsule 110 and gripper head 112. In the advanced position, the distal end of gripper backplate 122 may contact graft material 126, and further, apply force on graft material 126 so that graft material 126 is grasped between gripper backplate 122 and gripper head 112.

In the stage shown in FIG. 4D, a steerable laser catheter 102 may be introduced and tracked through gripper capsule and head 110 and 112. Laser catheter includes laser tip 130 at its distal end. While the steerable laser catheter 102 is being introduced through griper capsule and head 110 and 112, flexible distal portion 104 of steerable laser catheter 102 may be in undeflected position to facilitate tracking of the laser catheter. Once the laser catheter is tracked such that laser tip 130 is situated at distal portion 106 of steerable laser catheter 102, steerable laser catheter 102 may be transitioned from the unflexed position to the flexed position. Steerable catheter 102 may be flexed into a flexed position that creates a desired radius for the cutting operation of in-situ fenestration system 100.

In the stage shown in FIG. 4E, distal portion 106 (e.g., protective tip) of steerable laser catheter 102 is advanced in a proximal direction through graft material 126 while gripping backplate 122 and gripping head 112 grip graft material 126. In one or more embodiments, distal portion 106 advances as the gripper capsule and head 110 and 112 of the steerable gripper catheter remains stationary. Advancing distal portion 106 of steerable laser catheter 102 creates a gap between distal portion 106 and flexible distal portion 104. As shown in FIG. 4E, the length of the gap includes the thickness of graft material 126.

In the stage shown in FIG. 4F, laser tip 130 of the laser catheter is advanced into the gap created by distal portion 106 in the previous stage. As shown in FIG. 4F, laser tip 130 is aligned with graft material 126. Laser tip 130 is spaced apart a distance from longitudinal axis of steerable laser catheter 102 by a distance. The distance is representative of a radius of the fenestration created by a later cutting operation of in-situ fenestration system 100.

In the stage shown in FIG. 4G, laser tip 130 is energized to form a laser pulse. The laser pulse may be activated to an energy level corresponding to the amount of energy for removing graft material 126. Once laser tip 130 is energized, a rotational force is applied to a proximal portion of steerable laser catheter 102 to rotate laser tip 130 along a circumference having the radius described in connection with FIG. 4F. The laser tip 130 may rotate 360 degrees to form a continuous cutting path along the circumference from the laser pulse, thereby forming circular-shaped fenestrated material 128 in graft material 126.

In the stage shown in FIG. 4H, the fenestrated material 128 is removed by in-situ fenestration system 100. Steerable laser catheter 102 is moved into an undeflected position and retracted. Gripper capsule and head 110 and 112 of the steerable gripper catheter is retracted relative to graft material 126 and fenestration material 128 is evacuated. As they are grasping fenestrated material 128, gripper head 112 and gripper capsule 110 may be collectively removed from the patient's vasculature with steerable gripper catheter 52. In one or more embodiments, steerable catheter 102 is removed from the patient's vasculature with flexible distal portion 104 in an unflexed position.

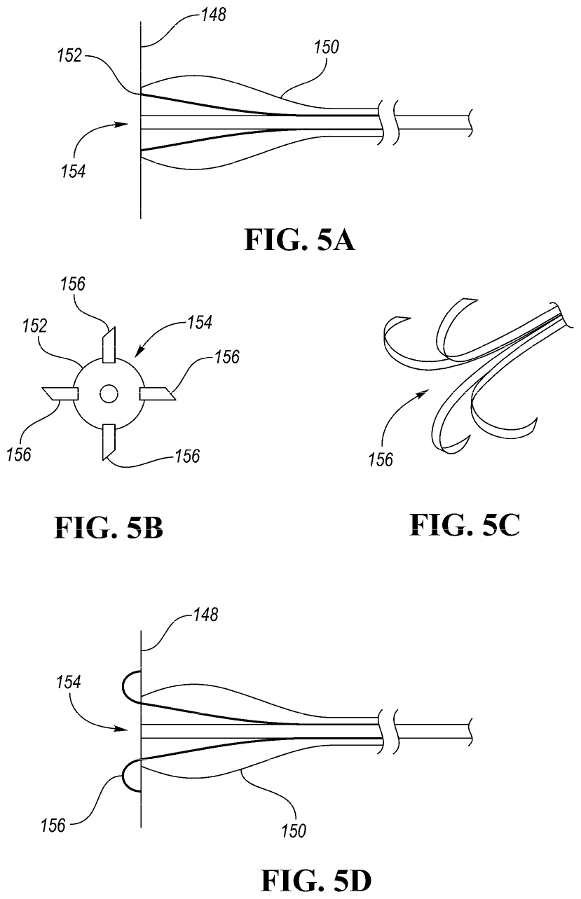

FIGS. 5A through 5D depict an alternative graft material gripping mechanism according to one or more embodiments for gripping graft material 148. FIG. 5A depicts a schematic, side view of gripping capsule 150 including a channel 152 sized to fit distal region 154 of a laser catheter. FIG. 5B is a schematic, end view of channel 152 and distal region 154 situated therewithin and gripping tines 156. FIG. 5C depicts an isolated, perspective view of gripping tines 156. FIG. 5D depicts a schematic, side view of gripping capsule 150 with gripping tines 156 in an extended position beyond graft material 148. Gripping tines 156 may be formed of a shape memory material such as Nitinol. When gripping tines 156 are advanced relative to gripping capsule 150, gripping times 156 expand outward to a pre-determined shape to align with the distal end of gripping capsule 150. Graft material 148 is configured to be gripped between gripping capsule 150 and gripping tines 156. This gripping operation pulls distal end (including a laser tip) of distal region 154 into contact with graft material 148. Once in contact, the laser tip may be energized to cut graft material 148. Gripping tines 156 may be configured to dilate a hole formed by a laser tip of the laser catheter. Pulling back on gripping tines 156 may be used to determine if the fenestration material has been fully excised. Torque feedback from the rotating laser catheter may be used to determine whether the cutting operation is proceeding as desired. The torque may be modulated to ensure proper cutting speed at the laser exit port.

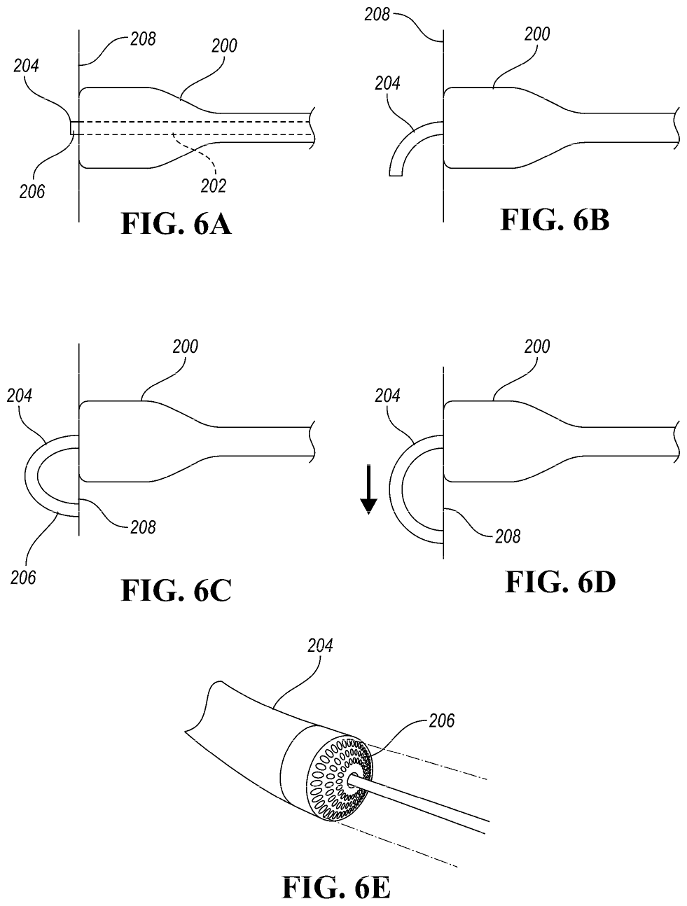

FIGS. 6A through 6E depict an embodiment with an articulating laser catheter used to perform a cutting operation of an in-situ fenestration process. FIG. 6A is a schematic, side view of gripping capsule 200 defining a channel 202 configured to track laser catheter 204 therethrough. As shown in FIG. 6A, laser catheter 204 has straight orientation where laser tip 206 is configured to pierce graft material 208. FIG. 6B depicts laser tip 206 of laser catheter 204 partially advanced beyond graft material 208 in an articulated position. FIG. 6C depicts laser tip 206 of laser catheter 204 further articulated such that laser tip 206 is facing (e.g., is perpendicular to) and contacts graft material 208. FIG. 6D depicts laser catheter 204 rotating such that laser tip 206 forms a circular path around graft material 208. Laser tip 206 may be energized before the rotating operation such that the laser tip 206 cuts through graft material 208 during the rotating operation. FIG. 6E depicts a perspective view of laser tip 206 of laser catheter 204.

The in-situ fenestration system of one or more embodiments may be delivered via femoral or radial access into an implanted graft and to a branch blood vessel using a steerable catheter system. In one or more embodiments, the in-situ fenestration system may utilize a combination of a guidewire, a vacuum, a mechanical grip, and a laser energy source to access, hold, cut, cauterize (e.g., melt or fuse), and remove fenestrated graft material. The delivery system of the in-situ fenestration system may include a steerable hollow catheter equipped for aspiration. The lumen of the steerable catheter is configured to permit the delivery of needle, guidewire, and laser components to a branch vessel location, while providing a protected area to perform a laser cut and to shield the body from gas bubbles. The in-situ fenestration system of one or more embodiments facilitates access to multiple geometries and anatomies by accounting for rotational and axial alignment to treat a greater patient population.

The in-situ fenestration system may be deployed procedural using one or more of the following steps. Access to a patient's vasculature may be gained through a femoral access site and a steerable gripper catheter 52 may be tracked to a branch vessel location using Fluro and/or echo guidance. Catheter flex may be applied to contact graft material with the distal tip of the steerable gripper catheter 52 and orient the distal tip perpendicular to the graft wall at a desired branch vessel location. The desired location may be determined by pre-implanted stents within the renal arteries. At this point, a gripping mechanism is activated to pierce the graft material and to hold the graft material wall to a laser cutting head. A laser-equipped system may be guided through a catheter lumen to a point of contact with the graft material to begin a cutting operation. A laser is energized/initiated to pierce the graft material wall. The laser guide is advanced through the graft material. Aspiration may then be initiated with aspiration port of the steerable gripper catheter 52. The steerable laser catheter may be advanced to obtain a desired cutting diameter. Laser power may then be initiated, and the laser guide may be rotated using handle controls. The laser head and gripper capsule may then be retracted to evacuate the cut graft material from the patient's vasculature.

One or more benefits of one or more embodiments include the following. An active steering system allows for precise fenestration positioning, both in an axial and radial direction. The graft material grasping methods of one or more embodiments provides improved placement and/or cutting precision control with reduced likelihood of fabric embolization after cutting. The laser energy used in one or more embodiments is configured to provide low impact graft penetration, with a short focal length to reduce/eliminate damage to the graft frame and patient tissue. The laser powered cutting may cauterize (e.g., melt or fuse) the graft material to reduce or eliminate frayed ends and embolic material associated with mechanical cutting. In one or more embodiments, vacuum aspiration coupled with laser activation improves the capture of bubbles, graft material, and/or any nano fabric particles that may escape cauterization during one or more laser pulses.

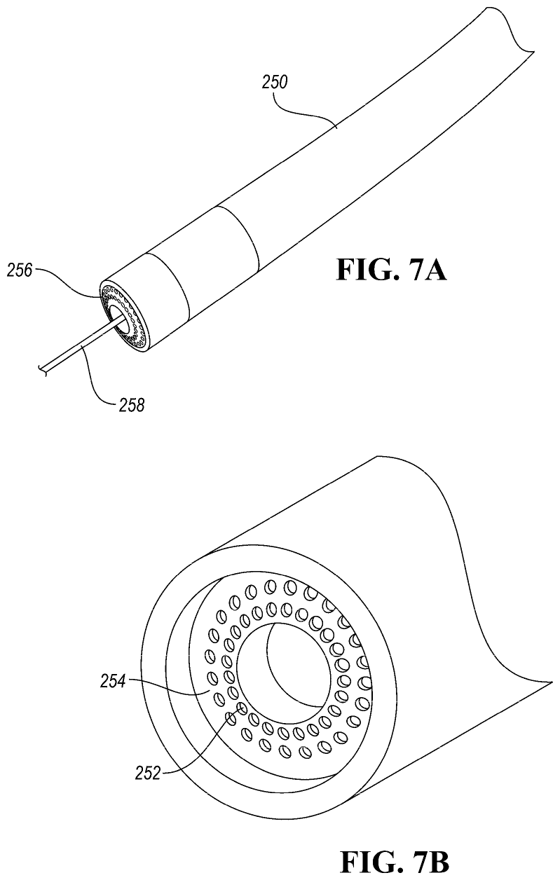

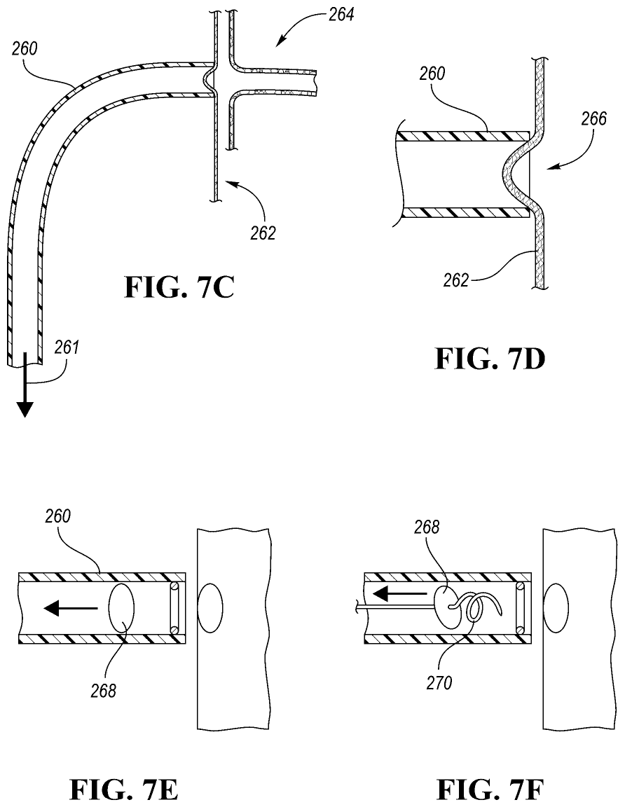

FIGS. 7A and 7B depict distal portion 250 of a laser catheter having first and second rings 252 and 254 of discrete ports (circular ports as shown in the figures) for laser light at distal end 256. The laser catheter may have a lumen configured so that the laser catheter may track along guidewire 258. First and second rings 252 and 254 are configured to deliver first and second fixed rings of light. As shown in FIG. 7B, first and second rings 252 and 254 are recessed within the laser catheter. This arrangement may offer protection from the patient's vasculature, due to the laser pulses being generated interior of the laser catheter.

FIGS. 7C through 7F depict an alternative embodiment where steerable catheter 260 is configured to impart a vacuum (signified by arrow 261) to grip graft material 262 aligned with the ostium of a branch vessel 264. FIG. 7D depicts gripped portion 266 of graft material 262 formed by imparting a vacuum condition within steerable catheter 260. The diameter of the fenestration may be controlled by the amount of material in gripped portion 266. As shown in FIG. 7E, fenestrated material 268 is aspirated by the vacuum within steerable catheter 260. Alternatively, coil 270 or a balloon may be used to excise fenestrated material 268.

In one embodiment, an in-situ fenestration system is disclosed. The in-situ fenestration device includes first and second inner catheters where the second inner catheter is disposed within the lumen of the first inner catheter. The first inner catheter may include a proximal cutter at a distal end thereof. The second inner catheter may include a distal cutter at a distal end thereof. The proximal cutter and the distal cutter cooperatively configured to cut an in-situ fenestration in graft material of a stent graft.

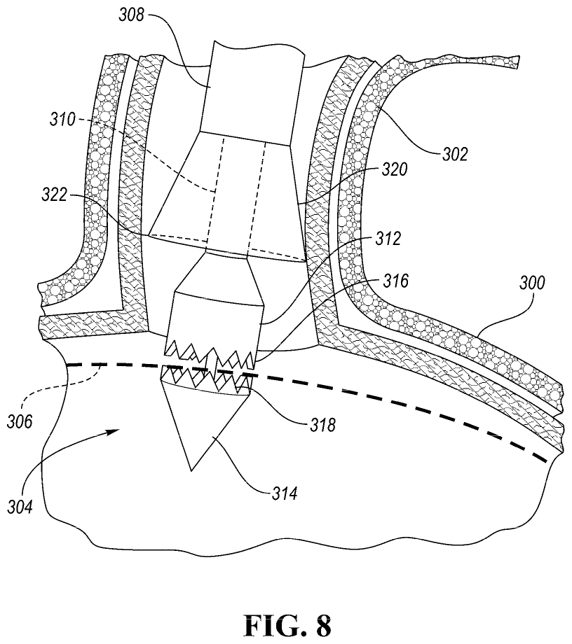

FIG. 8 depicts a schematic, cross section view of aortic arch 300 with branch vessel 302 branching therefrom where in-situ fenestration device 304 is deployed to form a fenestration in the graft material of stent graft 306. In-situ fenestration device 304 includes outer catheter 308 and inner catheter 310 situated therein. Inner catheter 310 may track along a guidewire (not shown) within outer catheter 308. Proximal base 312 of in-situ fenestration device 304 extends from and is connected to inner catheter 310. Inner catheter 310 may be a steerable catheter configured to be manipulated into a desired position for proximal and distal cutters 316 and 318 (as described below). The steerable catheter may limit or eliminate damage to the stents of stent graft 306 when creating an in-situ fenestration. The steerable catheter may also limit or eliminate damage to the sutures attaching the stents of stent graft 306 to the graft material.

In-situ fenestration device 304 also includes distal tip 314 (e.g., nose cone). Proximal base 312 and distal tip 314 may be directly or indirectly connected to each other. Proximal base 312 and distal tip 314 may be configured for movement relative to each other along a longitudinal axis of proximal base 312 and distal tip 314. Proximal base 312 and/or distal tip 314 may be spring loaded to create a punching movement of proximal cutters 316 and/or distal cutters 318 to create a fenestration and lock the cut graft material therebetween to remove it from the patient's vasculature.

Proximal base 312 includes proximal cutters 316 situated on the distal edge of proximal base 312. Distal tip 314 includes distal cutters 318 situated on the proximal edge of distal tip 314. Proximal cutters 316 and/or distal cutters 318 may include a sawtooth, triangular, or zigzag pattern. Such pattern is configured to limit the fraying of the graft material of stent graft 306. The cutters may limit fraying in a manner similar to that of pinking shears used to cut woven fabric with a triangular pattern. The angled cuts may cut along the bias of the fabric (e.g., not along the lengthwise or crosswise grain of the fabric). For a woven stent graft material, a similar cutting style may also reduce fraying of the graft material edge when cut. Proximal cutters 316 and/or distal cutters 318 may be expandable from a delivery configuration to an expanded configuration. FIG. 8 shows proximal cutters 316 and distal cutters 318 in the expanded configuration. Proximal cutters 316 may be configured to punch toward distal cutters 318 to create a fenestration in graft material situated between proximal cutters 316 and distal cutters 318. Distal tip 314 may be used to capture separated fenestrated graft material.

In one or more embodiments, an initial cut into the graft material may be made by a device configured to extend from distal tip 314. For instance, an initial cut may be made by a sharpened or pointed tip (e.g., a conical needle) or a blade extending from a lumen inside distal tip 314 of in-situ fenestration device 304. The sharpened or pointed may have a radiopaque marker (e.g., an “L” shaped radiopaque marker) for orientation purposes.

As shown in FIG. 8, embolic protection device 320 extends from the distal edge of outer catheter 308. Embolic protection device 320 is shown in an extended configuration. In one or more embodiments, embolic protection device 320 is retractable into a distal portion of outer catheter 308 into a retracted configuration. Embolic protection device 320 includes distal rim 322, which may be formed of a shape memory material (e.g., Nitinol material) configured to help expand embolic protection device 320 from the retracted configuration to the extended configuration upon advancing embolic protection device 320 relative to the distal edge of outer catheter 308. As shown in FIG. 8, embolic protection device 320 expands into the extended configuration behind proximal base 312. The extended configuration of the embolic protection device 320 may be sized to rest against an inner wall of branch vessel 302. Embolic protection device 320 may include a mesh, such as a Nitinol or polymer mesh, or other material capable of capturing embolic material and preventing it from flowing through or past the embolic protection device and downstream therefrom.

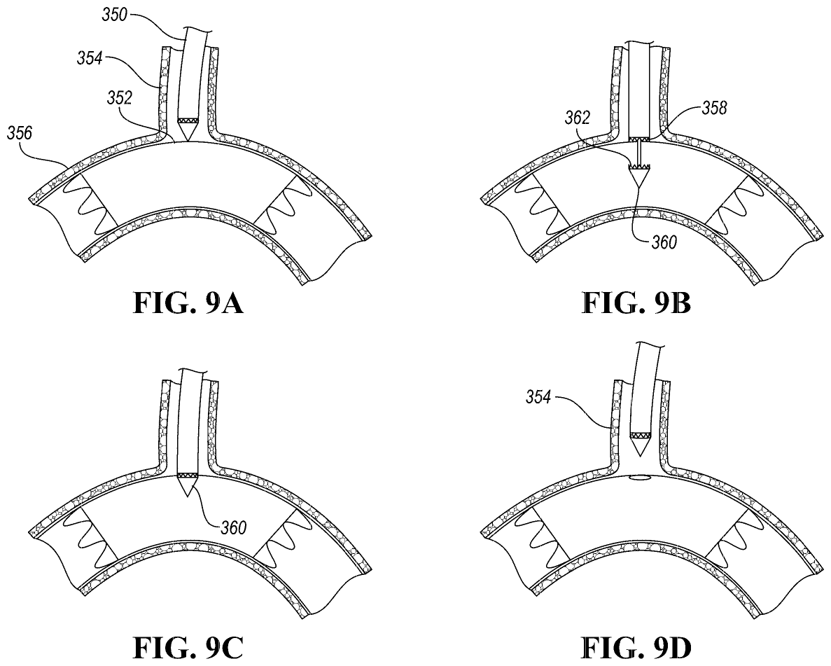

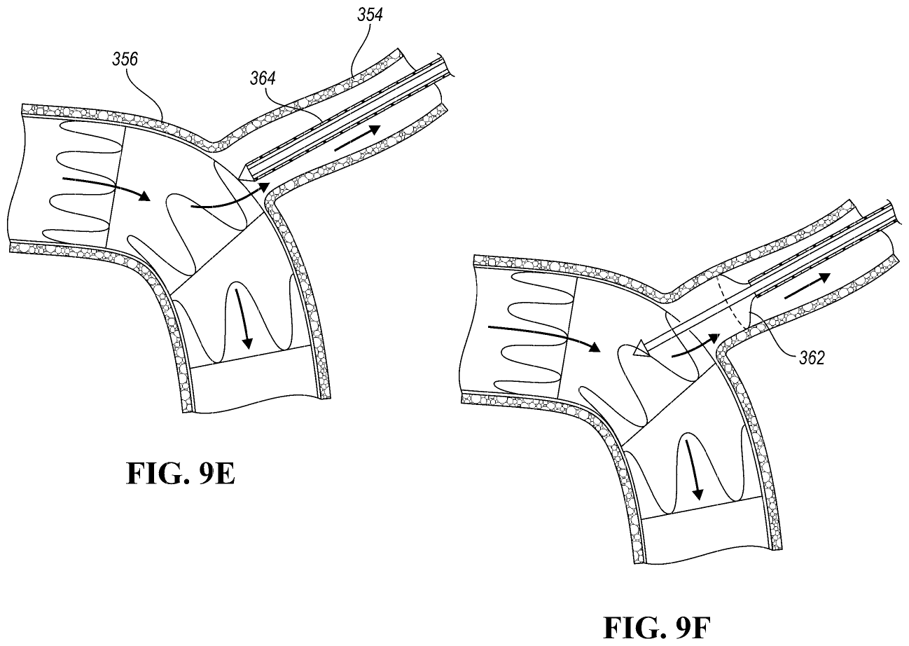

FIGS. 9A through 9D depict schematic, side views of in-situ fenestration device 350 performing a cutting operation on the graft material of stent graft 352. As shown in FIG. 9A, in-situ fenestration device 350 is tracked via branch vessel 354 (e.g., subclavian vessel) branching from aortic arch 356 into a desired position where in-situ fenestration device is aligned with a desired fenestration site on the graft material of stent graft 352. Fluro and/or left anterior oblique (LAO) viewing angle may be used to position device in its desired position.

As shown in FIG. 9B, in-situ fenestration device 350 includes proximal base including proximal cutters 358 and distal tip 360 including distal cutters 362. An initial cut into graft material of stent graft 352 may be made with a needle or other device configured to protrude from distal tip 360. The initial cut may be made by the distal tip or by an accessory device prior to tracking of the in-situ fenestration device 350. Once the initial cut is made, distal tip 360 is advanced beyond the graft material such that the graft material at the desired fenestration site is situated between proximal cutters 358 and distal cutters 362.

As shown in FIG. 9C, distal tip 360 is retracted such that proximal cutters 358 and distal cutters 362 contact each other to cut a fenestration at the desired location. The fenestrated material is captured between distal tip 360 and the proximal base. While FIG. 9C shows the distal tip 360 retracting to create the contact, in another embodiment the proximal base may be advanced relative to the distal tip 360 to create the contact between the proximal cutters 358 and distal cutters 362.

As shown in FIG. 9D, in-situ fenestration device 350 is pulled back through branch vessel 354 to remove the fenestrated material. Embolic protection device 362 (as described in connection with FIGS. 9E and 9F) is retracted into outer catheter 364. In-situ fenestration device 350 is then removed from the vasculature of the patient.

FIGS. 9E and 9F depict schematic, side views of in-situ fenestration device 350 and deployment of embolic protection device 362 therefrom. The arrows shown in FIGS. 9E and 9F depict blood flow through aortic arch 356 and branch vessel 354. In FIG. 9E, embolic protection device 362 is in a delivery configuration. In FIG. 9F, embolic protection device 362 is in a deployed configuration. In the delivery configuration, the shape of embolic protection device 362 is flattened or crimpled into a distal portion of the lumen of outer catheter 364 of in-situ fenestration device 350. Embolic protection device 362 may have Nitinol or other shape memory material sewn into the rim of embolic protection device 362. Before creation of an initial cut (e.g., between the operations shown in FIGS. 9A and 9B), embolic protection device 362 may be deployed into the deployed configuration by retracting outer catheter 364. The Nitinol or other shape memory material expands the cone shape of embolic protection device 362 such that embolic protection device 362 seats against the interior wall of branch vessel 354. In the deployed position, embolic protection device 362 is configured to catch any debris (e.g., freed fabric material from the graft) from creation of the fenestration. As shown in FIG. 9F, embolic protection device 362 in the deployed configuration has a flared distal portion aiding in the collection of any debris caused by the creation of the fenestration.

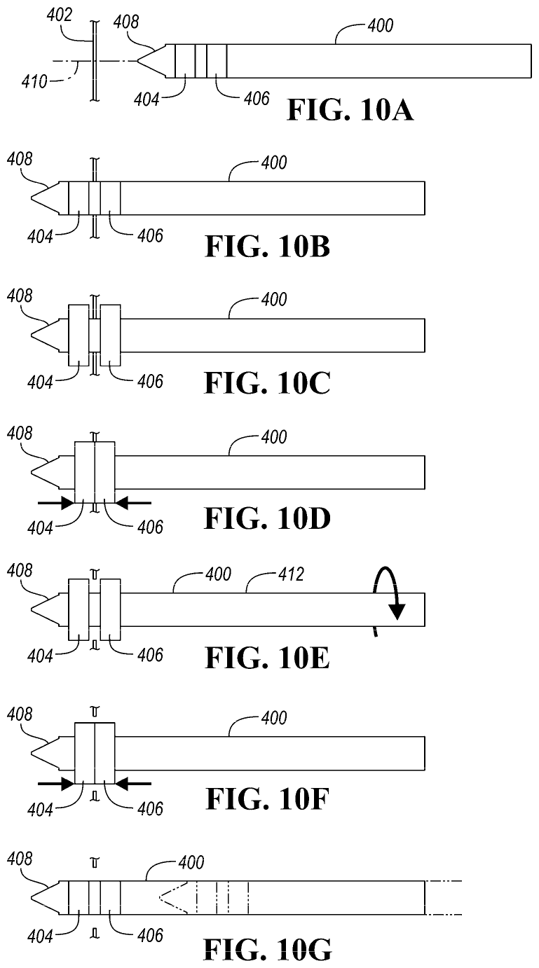

FIGS. 10A through 10F depict schematic, side views of in-situ fenestration device 400 and graft material 402 in relation to procedural steps for implementing in-situ fenestration device 400. In-situ fenestration device 400 includes distal cutters 404 and proximal cutters 406. Distal cutters 404 are carried on distal tip 408 of in-situ fenestration device 400. Proximal cutters 406 may be carried on a proximal base. Distal tip 408 and the proximal base are configured for relative movement between each other.

As shown in FIG. 10A, guidewire 410 extends into graft material 402 to form an initial cut into graft material 402. Distal tip 408 is configured to advance through the initial cut along guidewire 410.

As shown in FIG. 10B, in-situ fenestration device 400 is located such that distal cutters 404 and proximal cutters 406 are on either side of graft material 402. Distal cutters 404 and proximal cutters 406 are in a delivery configuration as shown in FIG. 10B. In the delivery configuration, the outer diameter of distal cutters 404 and proximal cutters 406 correlate (e.g., are equal to) the nominal outer diameter of catheter 412 of in-situ fenestration device 400, respectively.

As shown in FIG. 10C, distal cutters 404 and proximal cutters 406 are transitioned from the delivery configuration having a delivery diameter to a deployment configuration having a deployment diameter greater than the delivery diameter. Distal cutters 404 and proximal cutters 406 may be changed into a desired deployment diameter by a clinician actuating (e.g., turning) a control handle operatively connected to distal cutters 404 and proximal cutters 406. The control handle may simultaneously increase the diameter of distal cutters 404 and proximal cutters 406. In another embodiment, the control handle may independently increase the diameter of distal cutters 404 and proximal cutters 406. The deployment diameter of distal cutters 404 and proximal cutters 406 may be equal.

As shown by the arrows in FIG. 10D, distal cutters 404 and proximal cutters 406 are brought together to cut graft material 402 at a fenestration site by a shearing operation (e.g., scissor-like action). Distal cutters 404 and proximal cutters 406 may be brought together by axially translating one or both of distal cutters 404 and proximal cutters 406.

As shown in FIG. 10E, distal cutters 404 and proximal cutters 406 are separated, and then distal cutters 404 and proximal cutters 406 may be rotated to cut graft material not cut due to gaps between the blades of distal cutters 404 and proximal cutters 406. The arrow shown in FIG. 10E depicts rotational movement of catheter 412 of in-situ fenestration device 400, thereby rotating distal cutters 404 and proximal cutters 406, which are mechanically connected to catheter 412. In another embodiment, one or both of distal cutter 404 and proximal cutters 406 are rotated relative catheter 412. Rotation of the cutters may not be necessary and may depend on the configuration of the cutters. If there are gaps between adjacent cutting elements (e.g., saw teeth), rotation may be used to ensure that a complete cut is made.

As shown in FIG. 10F, distal cutters 404 and proximal cutters 406 are brought together again, thereby performing a second shearing operation to cut any remaining uncut material around the perimeter of the fenestration. If a full cut was made in the step of FIG. 10E, then this step may be omitted.

As shown in FIG. 10G, distal cutters 404 and proximal cutters 406 are transitioned from the deployment configuration to the delivery configuration by collapsing the blades of distal cutters 404 and proximal cutters 406, thereby trapping the cut graft material therebetween. In-situ fenestration device 400 is then removed from the vasculature of the patient, where distal cutters 404 and proximal cutters 406 have the delivery diameter, which may be equal to the diameter of catheter 412.

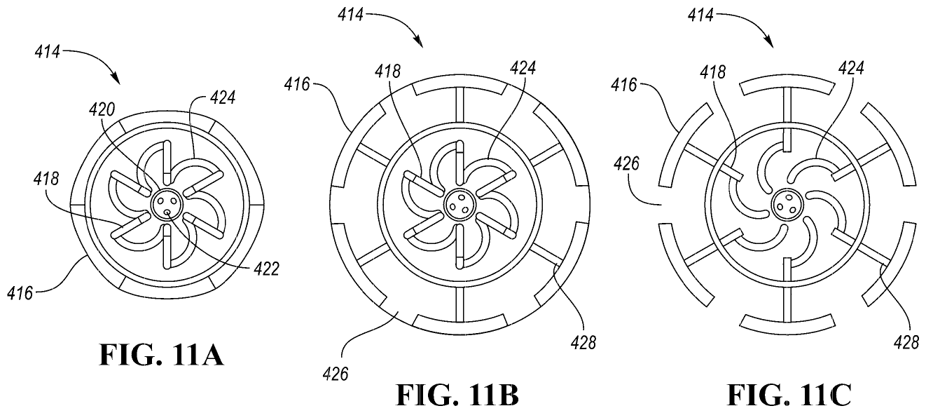

FIGS. 11A, 111B, and 11C are schematic, top views of cutter assembly 414 (e.g., distal cutters 404 or proximal cutters 406) in a delivery configuration, a first deployment configuration, and a second deployment configuration, respectively.

In FIG. 11A, lateral edges of blades 416 contact each other to form a continuous blade configuration. Arms 418 correspond to blades 416. Arms 418 and blades 416 are spaced apart from each other in the delivery configuration. Inner catheter 420 is centrally disposed to the continuous blade configuration. Inner catheter 420 includes wires 422 in mechanical communication with arms 418 through curved linkages 424. Wires 422 are in mechanical communication with an actuator (e.g., a turning knob) on the handle for catheter 412. The actuator is configured to be actuated to transfer mechanical movement of the actuator to outward, radial movement of arms 418 through wires 422 and curved linkages 424. The expanding mechanism allows a clinician to increase a diameter of the fenestration from a catheter handle.

As shown in FIG. 11B, arms 418 contact inner surfaces of blades 416 to extend the blades radially outward into the first deployment configuration such that the lateral edges of blades 416 are not contacting each other thereby forming gaps 426 therebetween. In the first deployment configuration, arms 418 are fully extended against blades 416. Distal ends 428 of arms 418 may engage the inner surface of blades 416 such that blades 416 retract radially inward when arms 418 are retracted radially inward.

As shown in FIG. 11C, blades 416 are shown in the second deployment configuration wherein the arms 418 are partially extended against blades 416. The actuator is configured to permit the clinician to return blades 416 from the second deployment configuration to the delivery configuration.

The catheter of the in-situ fenestration system may be available in multiple French sizes (e.g., 12, 13, 14, 15, 16, 17, or 18 Fr) to accommodate different, various access routes. The expandable cutters of one or more embodiments provide adaptability depending on specific anatomical specifications.

In one or more embodiments, the delivery system for the in-situ fenestration system may have three shafts (e.g., an inner shaft, a middle shaft, and an external shaft), where each shaft may be extended and/or retracted independently of each other via a handle. The inner shaft may run to a distal tip blade. A single pull wire (e.g., formed of stainless steel) may run through the inner shaft to allow the distal tip to be steered into position and angles to create a fenestration. The distal tip may also include a radiopaque marker (e.g., a radiopaque “L”) inside the distal tip so that a clinician can visualize the coaxility of the delivery system with the desired fenestration position and the branch vessel under Fluro. The middle shaft may terminate with the proximal base (e.g., punch section) of the cutter. The middle shaft may be connected to an actuator handle configured to allow a clinician to initiate a punch action to cut the graft material. The external shaft may be extended or retracted to have a flexible, steerable tip/sheath configured to enable the delivery system to be tracked atraumatically through a patient's anatomy. Further retraction of the external shaft allows for deployment of an expandable conical filter. The filter may be formed of a porous material such a porous polymeric membrane. The filter may be supported by a fine Nitinol frame secured around the rim of the conical filter. The Nitinol frame permits the edge of the conical filter to seat on the inside of a branch vessel proximal to a cutter. The delivery system may be flexible to enable tracking of the fenestration device.

In one embodiment, an in-situ fenestration system is disclosed. The in-situ fenestration system may include a fenestration catheter having a distal portion and a proximal tip separable from the distal portion. The fenestration catheter may include a distal element articulably connected to the distal portion and a proximal element articulably connected to the distal portion. The distal element and the proximal element may articulate from a delivery configuration to a deployment configuration. The profile of the distal and proximal elements in the deployment configuration are larger than a cross section profile of the fenestration catheter taken along a longitudinal axis thereof. The distal element and the proximal element in the deployment configuration may be configured to form a fenestration in graft material of a stent graft. The distal and proximal elements may have an elliptical profile.

One or more embodiments disclose a thermal based method to create fenestration in aortic stent grafts as part of in-situ fenestration procedures. One or more embodiments employs a thermal source. The thermal source may be a thermal source modified from a thermal source used as part of an endovascular method to create a percutaneous arteriovenous (AV) fistula for hemodialysis access in patients with end-stage renal disease. The power source used for an AV fistula may be a low power thermal energy source configured to cut the walls of two vessels and fuse the tissue together, creating an in-situ anastomosis between an artery and a vein. Further description of a device to form an in-situ anastomosis between an artery and a vein is provided in U.S. Pat. No. 9,452,015, which is incorporated herein in its entirety. The AV fistula device may be the ELLIPSYS device available from Avenu Medical, Inc. of San Juan Capistrano, California. The device in U.S. Pat. No. 9,452,015 may be used as a fenestration catheter to form in-situ fenestrations as described herein.

The in-situ fenestration device may have a catheter with a heating element surrounded by insulating material to limit the potential for incidental damage to aortic tissue. The catheter may be configured in an open configuration and a closed configuration. In the closed configuration, graft material being ablated may be trapped between the components of the catheter. A safety feature may be implemented into the system to prevent heating of the components if a stent is trapped between the closed catheter when the two components are near each other.

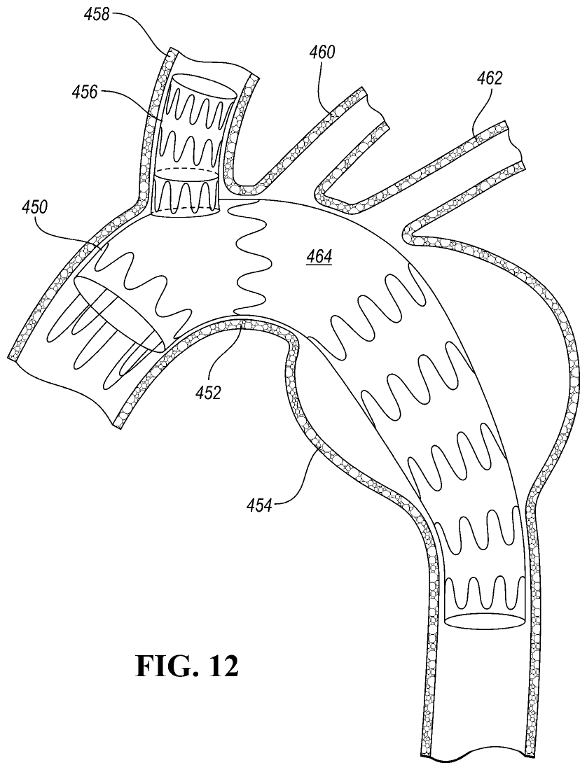

FIG. 12 depicts a schematic, side view of stent graft 450 deployed in aortic arch 452 to bridge aneurysm 454, and branch stent graft 456 disposed in brachiocephalic artery 458 branching from aortic arch 452. Left common carotid artery 460 and left subclavian artery 462 also branch from aortic arch 452. Stent graft 450 includes stentless region 464, which overlaps the ostia of left common carotid artery 460 and left subclavian artery 462. A stentless fenestration window may be built into stent graft 450 so that an in-situ fenestration may be performed without interaction with the stents of the stent graft. Alternatively, asymmetric stents may be employed to create stentless region 464.

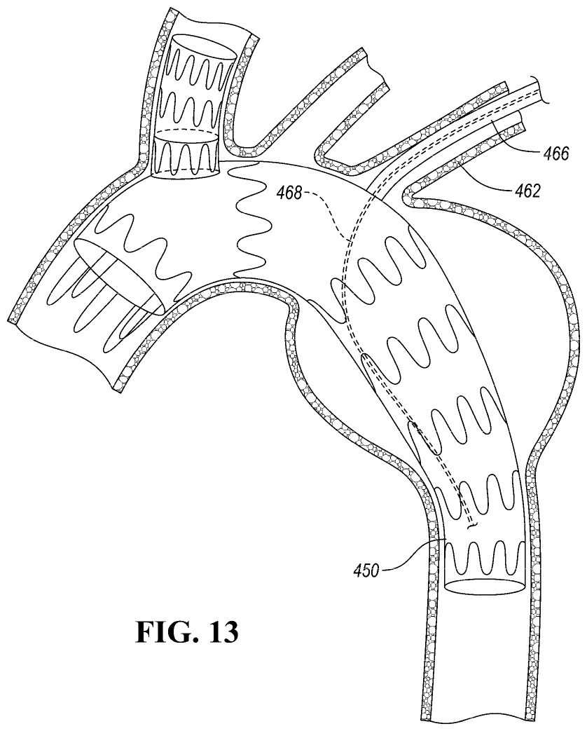

In one or more embodiments, a fenestration may be created in a retrograde manner using supra-aortic access. FIG. 13 depicts a schematic, side view of deflectable catheter 466 accessing stent graft 450 through left subclavian artery 462. Deflectable catheter 466 may have a centering mechanism configured to position a fenestration catheter perpendicular to the graft material of stent graft 450. Alternatively, a centering mechanism may be built into a thermal based fenestration catheter to permit centering of the catheter in a target vessel ostium. In one or more embodiments, tip deflection capabilities may be built into the catheter to allow for positioning in a target vessel ostium. Guidewire 468 may be advanced through graft material of stent graft 450 to obtain access to the aortic lumen. Alternatively, the fenestration catheter may have a sharpened end configured to traverse the graft material of stent graft 450 when advanced out of a protective sheath of the fenestration catheter. A triggering mechanism may be built into the fenestration catheter. The triggering mechanism may be configured to advance a needle or other form of sharpened tip across the graft material into the aortic lumen.

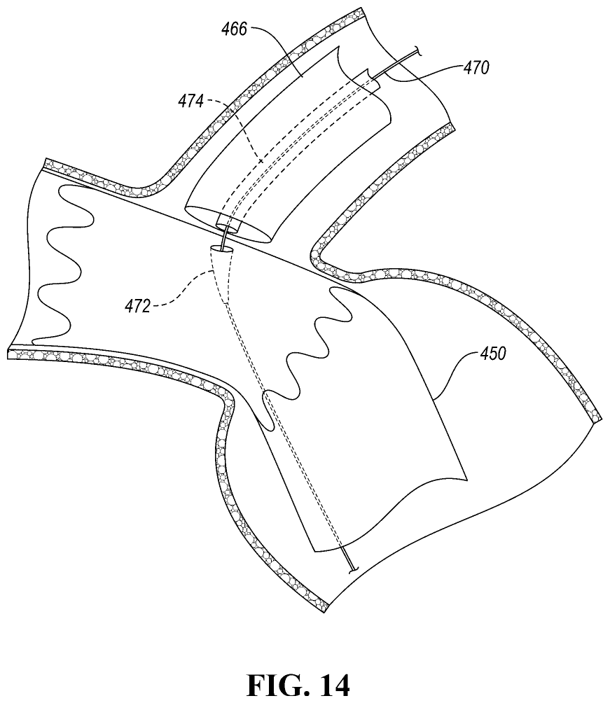

FIG. 14 depicts a schematic side view of fenestration catheter 470 advanced through deflectable catheter 466. Fenestration catheter 470 includes proximal portion 472 pushed through an initial fenestration in the graft material of stent graft 450 along guidewire 468. The initial fenestration may be formed mechanically with a sharpened or pointed tip or wire with or without additional energy being applied to the wires, such as ultrasound energy, RF energy, or other mechanism for translating energy where the cut is made. The initial fenestration may be dilated with a balloon to facilitate proximal portion 472 traversing the graft material. As shown in FIG. 14, proximal portion 472 of fenestration catheter 470 is separated and spaced apart from the distal portion 474 of fenestration catheter 470.

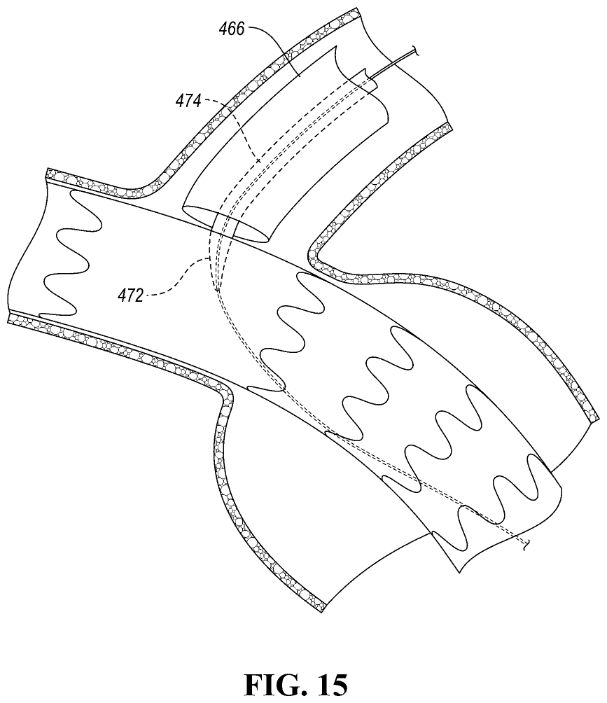

FIG. 15 depicts a schematic side view of fenestration catheter 470 where proximal portion 472 and distal portion 474 are in a closed configuration, thereby trapping graft material therebetween. As used herein, “proximal” may refer to closest to the heart or upstream, while “distal” may refer to farther from the heart or downstream. However, when referring to an operator, the descriptions may be reversed and proximal portion 472 could be considered a distal portion. The trapped graft material may be ablated by fenestration catheter 470. The closed configuration has a benefit of trapping the ablated material so that it can be safely removed from a patient's vasculature while diminishing an embolic risk. The size and shape of the fenestration may be optimized by creating a durable connection between the branch bridging stent and the aortic stent graft. The ratio of the fenestration size to the target vessel diameter may be optimized for a seal between the bridging stent and aortic stent interface. Once proximal portion 472 and distal portion 474 are in the closed configuration, energy may be applied to a create a thermal ablation. The temperature and/or duration of the power discharge may be optimized to create a fenestration without frayed or torn edges). This optimization may further be used to generate a fenestration without the generation of steam bubbles and/or char particles that may pose an embolic risk.

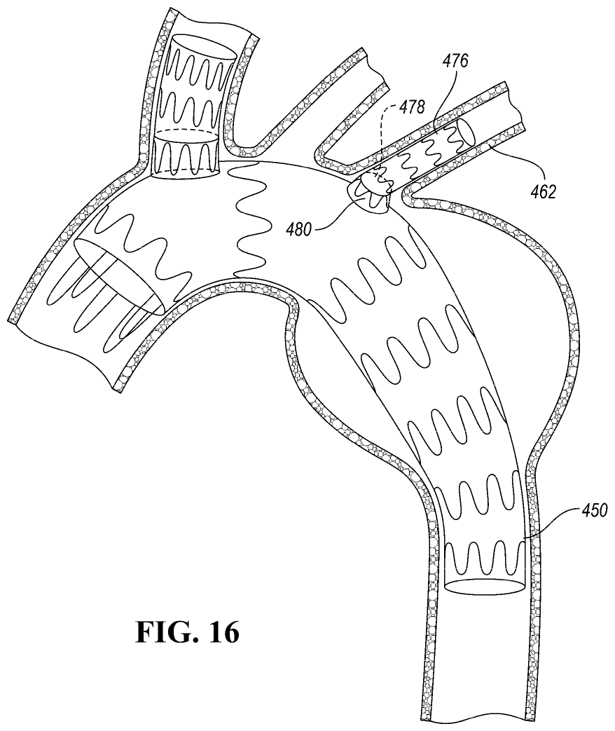

FIG. 16 depicts a schematic side view of branch stent graft 476 secured to fenestration 478 formed by a fenestration catheter (e.g., fenestration catheter 470). After formation of fenestration 478, the fenestration catheter is retracted through left subclavian artery 462, leaving a guidewire in place. Branch stent graft 476 may be tracked into place over the guidewire and deployed into fenestration 478. Proximal portion 480 (e.g., aortic lumen portion) may be post-dilated to create a flared end configured to improve the seal between branch stent graft 476 and stent graft 450 and to increase pullout force resistance. While FIGS. 13 through 16 depict a retrograde fenestration procedure initiated from supra-aortic access, the fenestration catheter of one or more embodiments may be used to create antegrade fenestrations from femoral access.

In one or more embodiments, a staged partial deployment of an aortic stent graft may be performed so that the stent graft does not fully expand to its nominal diameter using constraining ties and a trigger wire. The partial deployment may provide the advantage of creating space between the aortic wall and the aortic stent graft. This space may be used for canulation of the fenestration and the target branch vessel. The space provides a cushion so that a power discharge used to create the fenestration occurs away from sensitive vascular tissues. The partial deployment may be used with antegrade or retrograde access of the fenestration catheter. The constraining ties may be circumferential to uniformly limit the expansion of the aortic stent graft. Alternatively, the constraining ties may be asymmetrical and allow for an expansion of only an ostial facing portion of the aortic stent.

The following embodiment enables the provision of an enlarged fenestration without increasing the diameter of the fenestration device. Articulating elements (e.g., a proximal heating element and a distal backing element) may be incorporated into a flexible catheter configured to track over a guidewire. Tracking and operating over a guidewire enables the system to reach targets deeper into a patient's vasculature from femoral vessels. Branch vessels (e.g., celiac, superior mesenteric, and the renal arteries) may be accessed. The operation of the device and the articulating elements together create an elliptical fenestration in the graft material. This operation may be performed multiple times (e.g., two times) to create a relatively larger opening. For instance, two operations with 90 degrees of rotation may create an “X” fenestration pattern. The remaining flaps of graft material that reside between the elliptical cut outs may be moved outward from the center to enable a larger opening about the same diameter as the major axis of the elliptical shape.

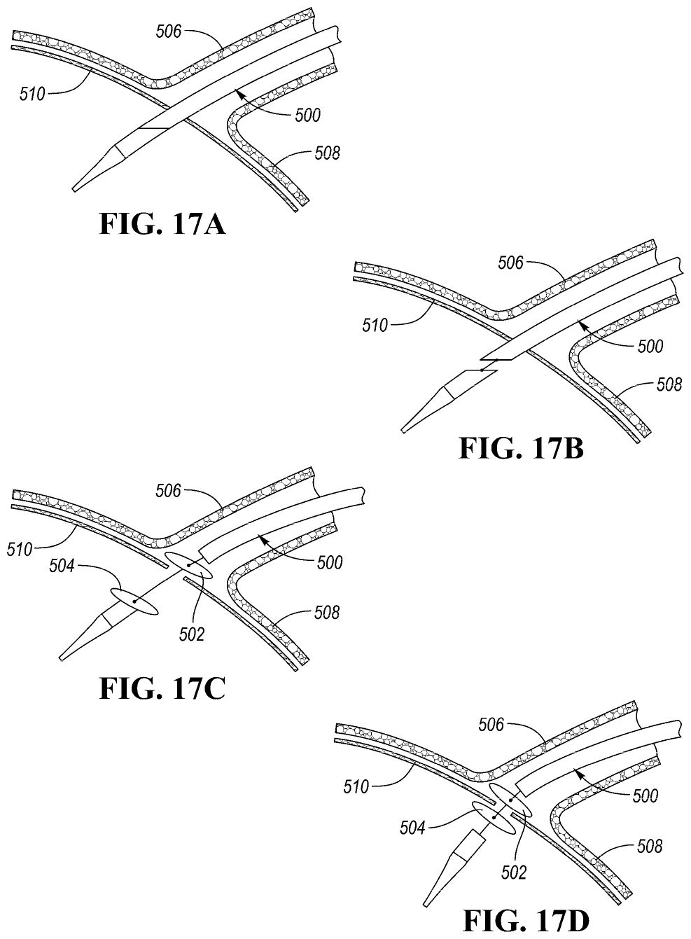

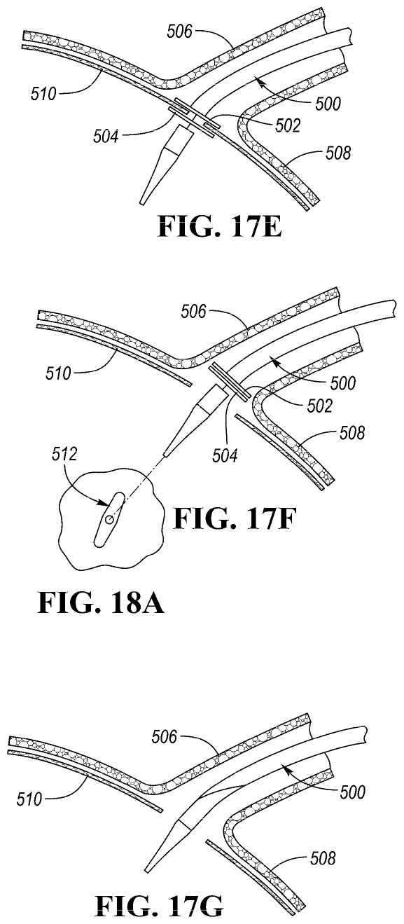

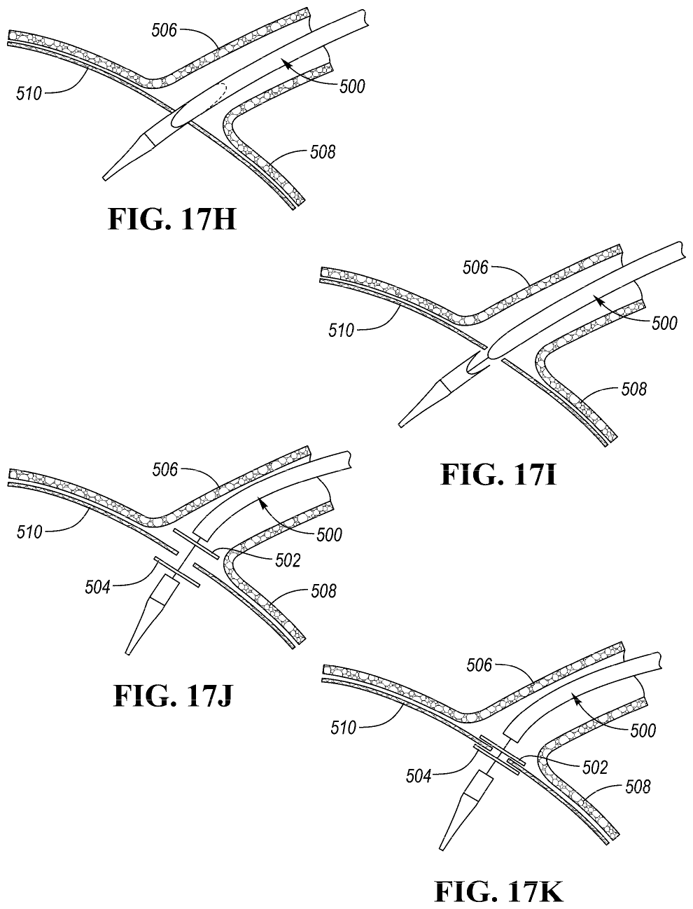

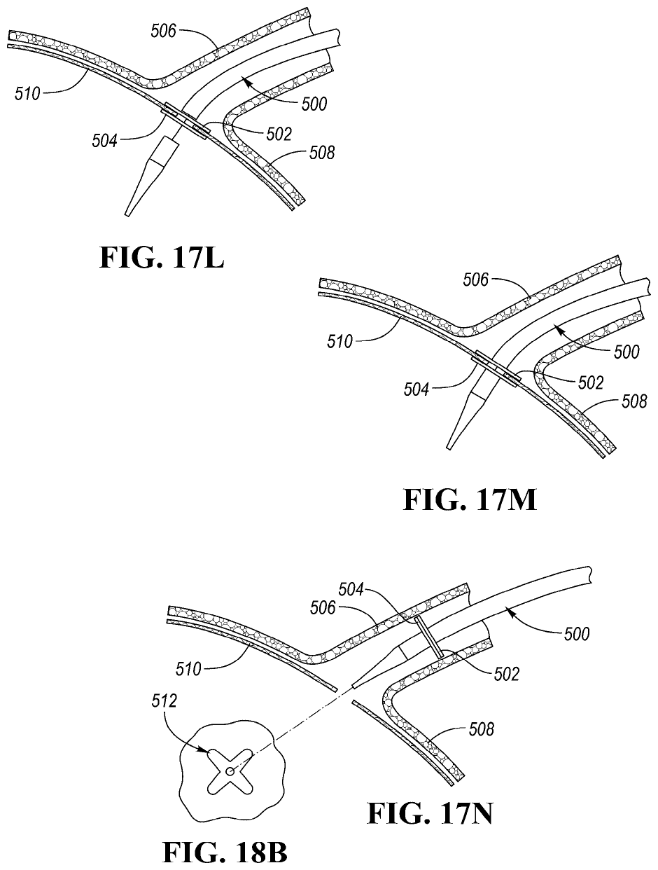

FIGS. 17A through 17N depict schematic side views of fenestration catheter 500 having first and second elliptical articulating elements 502 and 504 connected to fenestration catheter 500 in various stages of procedural deployment within branch vessel 506 branching from main vessel 508 having a deployed stent graft 510 therein. FIGS. 18A and 18B depict plan views of different stages of fenestration 512 formed by fenestration catheter 500.

Fenestration catheter 500 may be passed through a relatively small penetration in the stent graft wall as shown in FIG. 17A. In one or more embodiments, first elliptical articulating element 502 is oblique the longitudinal axis of fenestration catheter 500 in the delivery configuration. In one or more embodiments, second elliptical articulating element 504 is oblique the longitudinal axis of fenestration catheter 500 in the delivery configuration. Fenestration catheter 500 is configured to track guidewire 468. First elliptical articulating element 502 and/or second elliptical articulating element 504 may include first and/or second apertures configured to receive guidewire 468.

The elliptical articulating elements may be constrained within a relatively small profile (e.g., 3, 4, 5, 6, or 7 French) of the fenestration catheter during introduction, tracking and/or initial penetration of the stent graft wall. After penetration of the stent graft wall, the articulating elements may be separated as shown in FIG. 17B such that first elliptical articulating element 502 (e.g., distal backing element) is outside the stent graft, and second elliptical articulating element 504 (e.g., proximal heating element) resides on the stent graft interior. After each element is on respective sides of the stent graft material, pull/push wires may be actuated to rotate them separately from the circular profile of the catheter to expose their elliptical shape, as shown in FIG. 17C. The articulating elements continue to rotate about the catheter centerline until they are parallel to the graft material as shown in FIG. 17D. In the deployment configuration, first and/or second elliptical articulating elements 502 and/or 504 are parallel first and second portions of the graft material. In one or more embodiments, the first and second portions are the same graft material. In other embodiments, the first and second portions are different graft material.

A distal pull wire may be actuated (e.g., pulled) so that the distal backing element is pulled to the graft material and the proximal heating element is forced against the graft material, firmly opposing the distal backing element, as shown in FIG. 17E. Alternatively, the proximal heating element may be pulled to the graft material to force it against the distal backing element. With the two elements firmly opposed with graft material between them, a prescribed amount of current is passed through the proximal heating element to melt the graft material captured between the two elements. After the current has been delivered, the graft material between the elements is melted and dissociated from the body of the stent graft leaving an elliptical cut out as shown in FIG. 18A. To enlarge the opening, the pull/push wires are actuated in reverse order to stow the elliptical elements into the circular profile of the catheter as shown in FIGS. 17F and 17G.

In one or more embodiments, the catheter is rotated (e.g., 90 degrees) as shown in FIG. 17H, and the process of removing the graft material is repeated as shown in FIGS. 17I through 17N. The resulting opening created by the first and second operations leaves an “X” pattern of material removed from the stent graft as shown in FIG. 18B. The remaining flaps of graft material may be displaced without further damage to the graft material (e.g., ripping or tearing) to reveal an opening about the same diameter of the major axis of the elliptical articulating elements. While FIGS. 17 and 18 depict fenestrations formed of an elliptical shape, the fenestrations and the articulating elements may have other shapes. In one embodiment, the catheter may be withdrawn from the body between cutting steps to remove the graft material from the first fenestration step. The orientation of the catheter may be determined using one or more radiopaque (RO) markers such that the 90 degree rotation may be achieved despite withdrawing the catheter. In another embodiment, a second fenestration catheter may be used to create the second elliptical fenestration, with the first catheter being discarded.

The detailed description set forth herein includes several embodiments where each of the embodiments includes several components, features, and/or steps. For the avoidance of doubt, any component, feature, and/or step of one embodiment may be applied, mixed, substituted, matched, and/or combined with one or more components, features, and/or steps of other embodiments. Such resulting embodiments are expressly within the scope of this disclosure. For example, any systems and methods for locating a branch ostium of a branch vessel disclosed herein may be used in conjunction with any disclosed embodiments. Similarly, any systems, methods, or energy types for creating a fenestration (e.g., heat, laser, vibration, RF energy, blades/mechanical cutting) may be used in any disclosed embodiments. In any of the embodiments disclosed herein, following the creation of a fenestration the fenestration may be reinforced or strengthened by placing a stent or grommet like device in the fenestration. After a fenestration is created (and optionally reinforced), a branch stent graft may be tracked and deployed within the fenestration using a separate delivery system. The branch stent graft may extend within the fenestration and at least partially within a main lumen of the fenestrated stent graft and into branch artery (e.g., renal artery, celiac, SMA, BCA, LCC, LSA, etc.). The systems, methods, and devices disclosed herein may be used to make multiple fenestrations in a single stent graft, which thereafter each receive a branch stent graft.

While exemplary embodiments are described above, it is not intended that these embodiments describe all possible forms. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. An in-situ fenestration device comprising:
a fenestration catheter extending along a longitudinal axis; and
a proximal tip extending from the fenestration catheter,
the fenestration catheter including a first articulating element and a second articulating element, the first articulating element articulable the longitudinal axis from a delivery state to a deployment state, the second articulating element articulable about the longitudinal axis from the delivery state to the deployment state, and the first articulating element and the second articulating element in the deployment state are configured to form a fenestration in a graft material at a fenestration site of a stent graft.

2. The in-situ fenestration device of claim 1, wherein the first articulating element is a first elliptical articulating element, and the second articulating element is a second elliptical articulating element.

3. The in-situ fenestration device of claim 1, wherein the first and second articulating elements contact in the delivery state.

4. The in-situ fenestration device of claim 1, wherein the first and second articulating elements are spaced apart in the deployment state.

5. An in-situ fenestration device comprising:
a fenestration catheter extending along a longitudinal axis; and
a proximal tip extending from the fenestration catheter,
the fenestration catheter including a distal element articulable the longitudinal axis from a delivery state to a deployment state, the fenestration catheter including a proximal element articulable the longitudinal axis from the delivery state to the deployment state, the distal element having a distal element delivery cross sectional profile in the delivery state relative the longitudinal axis and a distal element deployment cross sectional profile in the deployment state relative the longitudinal axis, the distal element deployment cross sectional profile is larger than the distal element delivery cross sectional profile, the proximal element having a proximal element delivery cross sectional profile in the delivery state relative the longitudinal axis and a proximal element deployment cross sectional profile in the deployment state relative the longitudinal axis, the proximal element deployment cross sectional profile is larger than the proximal element delivery cross sectional profile, and the distal element and the proximal element in the deployment state are configured to form a fenestration in a graft material at a fenestration site of a stent graft.

6. The in-situ fenestration device of claim 5, wherein the distal element has a distal element elliptical profile and the proximal element has a proximal element elliptical profile.

7. The in-situ fenestration device of claim 5, wherein the distal element includes a distal heating element and the proximal element includes a proximal heating element, the distal heating element and the proximal heating element are configured to heat the graft material at the fenestration site of the stent graft to form the fenestration.

8. The in-situ fenestration device of claim 5, wherein the distal element and the proximal element contact in the delivery state and are spaced apart in the deployment state.

9. The in-situ fenestration device of claim 5, wherein the distal element is oblique the longitudinal axis of the fenestration catheter in the delivery state and/or the proximal element is oblique the longitudinal axis of the fenestration catheter in the delivery state.

10. The in-situ fenestration device of claim 5, wherein the distal element is parallel a first portion of the graft material in the deployment state and/or the proximal element is parallel a second portion of the graft material in the deployment state.

11. The in-situ fenestration device of claim 5, wherein the fenestration catheter is configured to track a guidewire, the distal element defines a distal element aperture configured to receive the guidewire, and the proximal element defines a proximal element aperture configured to receive the guidewire.

12. The in-situ fenestration device of claim 5, wherein the proximal tip is configured to push through an initial fenestration of the graft material.

13. A method of forming a fenestration in a graft material at a fenestration site of a stent graft, the method comprising:
delivering a fenestration device to the fenestration site, the fenestration device including a fenestration catheter extending along a longitudinal axis and a proximal tip extending from the fenestration catheter, the fenestration catheter including first and second articulating elements;
deploying the fenestration device at the fenestration site by positioning the first and second articulating element on first and second sides of the graft material and articulating the first and second articulating elements to form spaced apart first and second articulated elements;
contacting the spaced apart first and second articulated elements with the graft material to form contacting first and second articulated elements; and
applying heat to at least one of the contacting first and second articulated elements to disassociate the graft material at the fenestration site to form the fenestration from the stent graft.

14. The method of claim 13 further comprising returning the first and second articulating elements to a delivery state after the applying step.

15. The method of claim 13 further comprising removing the graft material at the fenestration site from a vasculature of a patient.

16. The method of claim 14 further comprising rotating the fenestration catheter after the returning step.

17. The method of claim 15 further comprising repeating the deploying, contacting, and applying steps after the rotating step to increase a size of the fenestration.

18. The method of claim 16, wherein a pattern of the fenestration is an “X” pattern.

19. The method of claim 17, wherein the fenestration site includes flaps of the graft material bordering the fenestration, and further comprising moving the flaps of graft material outward to further increase the size of the fenestration.

20. The method of claim 13, wherein the applying step includes delivering current to one of the first and second articulated elements.
